(19) United States
(12) Patent Application Publication
(10) Pub. No.: US 2022/0016279 A1
(43) Pub. Date: Jan. 20, 2022

(54) Disinfection Device and Related Systems and Methods

(71) Applicant: Dane Technologies, Inc., Maple Grove, MN (US)

(72) Inventor: Dan Johnson, Medina, MN (US)

(21) Appl. No.: 16/928,982

(22) Filed: Jul. 14, 2020

(51) Int. Cl. A61L 2/10; A61L 2/26

(52) U.S. Cl. CPC A61L 2/10; A61L 2202/20; A61L 2202/122; A61L 2/26

(57) ABSTRACT

Disclosed herein are various disinfection devices for disinfecting various objects. Such devices can include a device having a body, at least one light array disposed on an interior surface of the body, and a conveyor disposed within the body. Further disclosed are various methods of disinfecting various objects using such disinfection devices.

### FIELD

The various embodiments herein relate to disinfection devices, including automated disinfection devices, for use in various environments, including, for example, volume disinfection of multiple objects.

### BACKGROUND OF THE INVENTION

The ongoing threat of bacteria and viruses and resulting pandemics in the world today has increased the need to disinfect various items, including purchased items such as groceries and the like. Bacteria and virus pathogens cause many illnesses and infections, and such pathogens on an object or a surface provide a pathway to infection when a person contacts that object or surface.

Several known disinfection techniques have been used to reduce the risk of the spread of infectious disease.

One such technique is the use of disinfectant wipes. One disadvantage is that many surfaces do not lend themselves to be effectively disinfected using a wipe because of their design and/or structure.

Another known technique involves the use of a disinfectant solution that is sprayed, misted, or fogged on the surface or object to be disinfected. One disadvantage of this technique is that the surface or object becomes wet, and another is the high recurring cost.

A further technique is the use of UV light to disinfect surfaces, fluids, and air. One disadvantage is that the wavelength of most disinfection UV light has been documented to cause damage to human tissue, including skin and eyes.

There is a need in the art for improved disinfection devices and related methods.

### BRIEF SUMMARY

Discussed herein are various disinfection devices for disinfecting various objects and methods of disinfecting objects using such devices.

In Example 1, a disinfection device comprises a body comprising a bottom wall, two side walls coupled to the bottom wall, and a top wall coupled to the two side walls. The device further comprises at least one light array disposed on an interior surface of each of the two side walls and the top wall, and a conveyor disposed on the bottom wall.

Example 2 relates to the disinfection device according to Example 1, wherein the at least one light array comprises at least two UV light bulbs.

Example 3 relates to the disinfection device according to Example 1, wherein the at least one light array comprises a first shield disposed on a first side of the light array and a second shield disposed on a second side of the light array.

Example 4 relates to the disinfection device according to Example 1, wherein the at least one light array is moveable between a retracted position and an extended position.

Example 5 relates to the disinfection device according to Example 1, further comprising at least one protective structure disposed on the interior surface of each of the two side walls and the top wall.

Example 6 relates to the disinfection device according to Example 5, wherein the at least one protective structure is a bar disposed at a predetermined distance from the interior surface.

Example 7 relates to the disinfection device according to Example 1, wherein the conveyor comprises two conveyor rollers, and a conveyor belt disposed around the two conveyor rollers.

Example 8 relates to the disinfection device according to Example 7, wherein the conveyor belt comprises ridges disposed on an outer surface of the conveyor belt.

In Example 9, a disinfection device comprises a body comprising, a bottom wall, two side walls coupled to the bottom wall, and a top wall coupled to the two side walls. The device further comprises at least one light array disposed on an interior surface of each of the two side walls and the top wall, and the at least one light array comprises at least two UV light bulbs, a first shield disposed on a first side of the light array, and a second shield disposed on a second side of the light array. In addition, the device also comprises a conveyor disposed on the interior surface of the bottom wall or the top wall.

Example 10 relates to the disinfection device according to Example 9, wherein the at least one light array is moveable between a retracted position and an extended position.

Example 11 relates to the disinfection device according to Example 9, further comprising at least one protective structure disposed on the interior surface of each of the two side walls and the top wall.

Example 12 relates to the disinfection device according to Example 11, wherein the at least one protective structure is a bar disposed at a predetermined distance from the interior surface.

Example 13 relates to the disinfection device according to Example 9, wherein the conveyor is disposed on the interior surface of the bottom wall, the conveyor comprising two conveyor rollers, and a conveyor belt disposed around the two conveyor rollers.

Example 14 relates to the disinfection device according to Example 13, wherein the conveyor belt comprises ridges disposed on an outer surface of the conveyor belt.

Example 15 relates to the disinfection device according to Example 9, wherein the conveyor is attached to the interior surface of the top wall, the conveyor comprising a conveyor rail.

In Example 16, a method of disinfecting objects in an industrial or retail environment comprises positioning an object into a disinfection device, which comprises a body comprising a bottom wall, two side walls coupled to the bottom wall, and a top wall coupled to the two side walls. The device also comprises at least one light array disposed on an interior surface of each of the two side walls and the top wall. The method further comprises actuating the at least one light array to emit UV light onto the object for a calculated amount of time, and urging the object out of the disinfection device.

Example 17 relates to the method according to Example 16, wherein the positioning the object into the disinfection device further comprising inserting the object through an entrance and into an interior of the disinfection device.

Example 18 relates to the method according to Example 16, wherein the disinfection device further comprises a conveyor, wherein the urging the object out of the disinfection device further comprises urging the object out of the disinfection device via the conveyor.

Example 19 relates to the method according to Example 16, wherein the object is a retail shopping cart containing purchased items or the object is at least one article of clothing.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1A is a perspective view of a disinfection device, according to one embodiment.

FIG. 1B is a front view of the disinfection device of FIG. 1A, according to one embodiment.

FIG. 10 is a side view of the disinfection device of FIG. 1A, according to one embodiment.

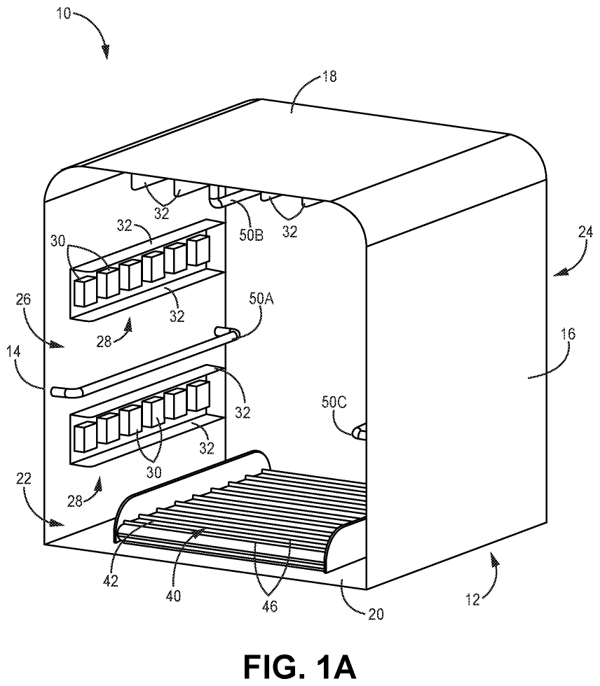

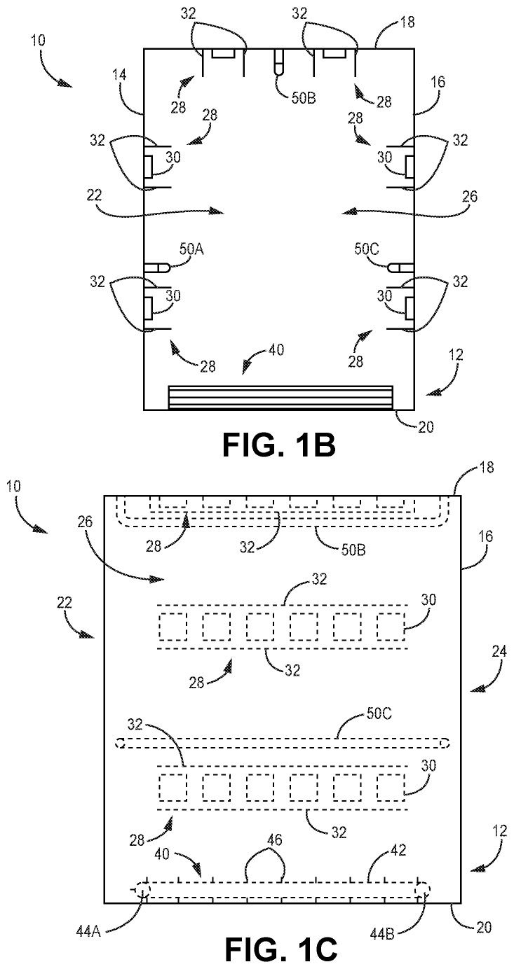

FIG. 1D is a top view of the disinfection device of FIG. 1A, according to one embodiment.

### DETAILED DESCRIPTION

The various embodiments herein relate to a disinfection device, and more specifically to a disinfection device that receives one or more object (serially) through one opening and allows the object(s) to pass through the device and exit out of another opening. In certain implementations, the device is automated via a conveyor such that the object(s) are placed on the conveyor and urged through the device via the belt. The device can have UV lights or any other type of disinfection media within the device that are used to disinfect each object as it passes through.

According to one exemplary embodiment as depicted in FIGS. 1A-1D, the disinfection device 10 has a body 12 that is made up of side walls 14, 16, a top wall 18, and a bottom wall 20. Further, the device 10 has a first opening (or “entrance”) 22, a second opening (or “exit”) 24, and an interior 26 defined by the walls 14, 16, 18, 20 and in communication with the openings 22, 24. In this specific implementation, the device 10 has UV light arrays 28 disposed in the interior 26 on the inner surfaces of both side walls 14, 16, and the top wall 18 as shown. In the specific embodiment depicted, there are two arrays 28 disposed on each of the walls 14, 16, 18. Alternatively, any number of arrays 28 can be disposed on each wall 14, 16, 18 in the interior 26 of the body 12. Each array 28 has at least two UV light bulbs 30. In this specific implementation, each array 28 has six light bulbs 30. Alternatively, each array 28 can have any number of bulbs 30.

In accordance with certain embodiments, the light arrays 28 are extendable or positionable. More specifically, the light arrays 28 can not only be disposed as shown against the inner surfaces of the walls 14, 16, 18, but also can be extended or otherwise moved away from the inner surfaces to some predetermined distance from the walls 14, 16, 18 (not shown), and thus closer to the object being urged through the device 10. That is, the position of each of the light arrays 28 is adjustable in relation to the walls 14, 16, 18. This positionable nature of the light arrays 28 allows for the arrays 28 to be adjusted to the size of the object(s) being urged through the device 10, thereby optimizing the position of the light arrays 28 in relation to the object(s) and hence the intensity of the UV light that is irradiating the object(s). The light arrays 28 can be extended any known distance away from the walls 14, 16, 18. In accordance with certain implementations, there are sensors (not shown) disposed on some portion of the device 10 to detect the distance between the object and the walls 14, 16, 18, thereby determining the general size of the object. That information can be transmitted to an onboard processor and utilized to actuate the light arrays 28 to extend a predetermined distance (based on the information from the sensors) toward the object, thereby optimizing the intensity of the UV light. Alternatively, the distance that the light arrays 28 extend from the inner surfaces of the walls 14, 16, 18 can be determined manually (by a user viewing the size of the object, for example).

In the implementation as shown, each of the light arrays 28 also have shields 32 disposed on both sides of the light bulbs 30. The shields 32 direct or “focus” the light emitted from the bulbs 30 in the desired direction. For example, in certain embodiments, the shields 32 reduce the risk of the UV light “escaping” from the device 10 and putting any person nearby at risk. Alternatively, the arrays 28 can have no shields.

Alternatively, instead of being positioned parallel to the length of the body 12 as shown, the light arrays 28 can instead be disposed such that the length of each array 28 is transverse to the longitudinal axis of the body 12. It is understood that the body 12 has a length that extends from the entrance 22 to the exit 24 of the body 12. Thus, while the arrays 28 as shown in the embodiment of FIGS. 1A-1D are disposed such that they are parallel to a longitudinal axis of the body 12, certain alternative embodiments as contemplated herein can have light arrays 28 that are positioned such that an axis of each such array 28 is transverse to the longitudinal axis of the body 12. In a further alternative, some of the arrays 28 can be parallel to the longitudinal axis, while others are transverse.

Each of the bulbs 30 can be a 24 volt bulb. Alternatively, the bulbs 30 can be any known bulbs have any known voltage, wattage, or size.

In one embodiment, the light bulbs 30 in the arrays 28 emit ultraviolet (“UV”) light. In certain more specific implementations, the bulbs 30 emit UV-C light, which has a wavelength ranging from 100 to 280 nm. Alternatively, the bulbs 30 can emit any known light that can disinfect objects and/or the ambient air.

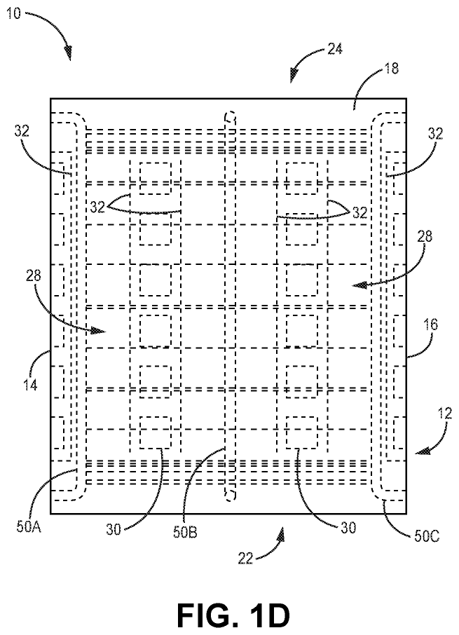

The device 10 implementation as shown in FIGS. 1A-1D also has a conveyor 40. Alternatively, the device 10 can have any known transport mechanism for conveying or otherwise urging an object through the device 10. The conveyor 40 has a belt 42 that is rotatably disposed around two rollers 44A, 44B (as best shown in FIG. 10) such that rotation of one or both of the rollers 44A, 44B causes rotation of the belt 42 around the rollers 44A, 44B, which causes any object disposed on top of belt 42 to be transported laterally along the conveyor 40. It is understood that the conveyor 40 can be any commercially available conveyor of the appropriate size. The conveyor 40 in certain embodiments is powered by an actuator (not shown), such as, for example, a motor. Alternatively, the conveyor 40 can be manually powered. In a further alternative, the device 10 can have no conveyor, and the object can be urged or otherwise transported through the device 10 by a user or via any other known means.

According to one specific embodiment as best shown in FIGS. 1A and 10, the conveyor belt 42 has ridges 46 formed, attached, or otherwise disposed on the outer surface of the belt 42 such that the ridges 46 will contact the object being urged through the device 10 by the belt 42 and thereby help to transport the object through the device 10. For example, in one embodiment, the object is a shopping cart containing groceries and/or other retail items (not shown), and as the belt 42 rotates and the cart is urged into contact with the belt 42, a ridge 46 will make contact with a rear portion of each of the two front wheels of the cart (not shown) and help to urge the cart through the device 10. Alternatively, the object can be any type of object that would benefit from disinfection, and the ridges 46 can help to urge any such object through the device 10. In a further embodiment, the belt 42 can have any known structure(s) (such as ribs, bars, hooks, etc.) on the outer surface thereof such that the structure(s) can be used to help urge the objects through the device 10.

In a further alternative, the conveyor 40 can be two different conveyors (not shown) disposed adjacent to each other (or a divided single conveyor) with a gap defined between the two conveyors (not shown). Thus, in such embodiments, it is possible to position a light array (similar to light arrays 28) in that gap to emit light upward from the position between the two conveyors (not shown).

In accordance with certain implementations, including the implementation of FIGS. 1A-1D, the device 10 can also have protective structures 50 (as best shown in FIG. 1B) attached to or otherwise disposed along the inner surfaces of the side walls 14, 16, and the top wall 18 to prevent any object passing through the device 10 from contacting and potentially damaging any of the light arrays 28. In the exemplary embodiment depicted, the protective structures 50 are protective elongate bars 50 that extend along the length of the inner surfaces of the walls 14, 16, 18 such that the bars 50 are substantially parallel with the length of the belt 42 as shown. Alternatively, the protective structures 50 can be any known structures that are disposed along the inner surfaces of the walls 14, 16, 18 and prevent any object to be disinfected from contacting and thus potentially damaging any of the light arrays 28 as it is urged through the device 10.

The device 10, in certain embodiments, can also have sensors (not shown) disposed at the entrance 22 and/or within the interior 26 of the body 12 to detect the presence of an object. The sensors (not shown) can be coupled to the actuator (not shown) such that when the sensors detect the presence of an object, a signal is transmitted to the actuator to activate the actuator, thereby actuating the conveyor to transport the object through the device 10. In one embodiment, the sensors (not shown) disposed at the entrance and within the interior 26 continue to transmit signals to the actuator so long as the object is disposed at the entrance 22 and/or at least partially disposed within the interior 26 such that the conveyor 40 continues to operate until the sensors no longer detect the presence of the object. Alternatively, any arrangement of sensors and/or other detection devices can be used to provide for automated actuation of the conveyor 40.

In a further alternative, the device 10 can also have one or more cameras and/or RFID readers disposed on or within the device 10 to capture information about the object. For example, in one embodiment in which the object is a shopping cart (not shown), the camera(s) and/or RFID reader(s) can be used to identify the items within the shopping cart and transmit that information to an onboard processor or wirelessly to a processor disposed elsewhere. As such, the information about the object/items can be collected for various purposes. In the shopping cart example, if the specific venue is a retail store, the information can be collected and compared to the purchase information/receipt relating to that specific cart to confirm that the items disposed within the cart were purchased, thereby helping to prevent theft. In addition, or alternatively, the information can be collected to track the purchasing behavior of the specific customer or customers as a whole (or both). In a further alternative, the information can be collected to help track inventory at the venue. In yet another alternative, the information can be collected for any known reason.

It is understood that, with a given UV light bulb output, the required disinfection dose is directly related to the distance of the bulb from the target/object surface and duration of the light being applied to that surface. For example, according to one embodiment, a 300 watt bulb can disinfect all or almost all of a surface positioned between about 6 and about 12 inches from the bulb while the conveyor 40 (and thus the object) is traveling at a rate of 1 foot per second. It is understood that any of these variables can be adjusted based on the adjustment of the other variables to achieve an equivalent level of success with respect to disinfection. For example, if either the distance or the bulb intensity changes for some reason, the speed of the conveyor 40 can be adjusted accordingly. Further, because the conveyor 40 speed, the light bulb output, and the distance between the bulbs and the object are known, those parameters can be set to ensure disinfection of the object such that any object that passes through the device 10 based on those parameters can be confirmed or “certified” to have been disinfected successfully.

According to one embodiment, an onboard processor or, alternatively, software that is incorporated into a system that is in communication with an onboard processor, can be used to determine either the optimal speed of the conveyor 40 and/or the optimal distance between the light arrays 28 and the object(s) assuming a specific bulb intensity. Further, if either the distance or the bulb intensity changes for some reason, the processor/software can be used to adjust the speed of the conveyor 40, or stop and start the conveyor 40, accordingly. As a result, the three parameters (conveyor speed, distance, and bulb intensity) can be controlled to ensure that and confirm that the object has been disinfected successfully. Any onboard processor is coupled to the light arrays 128 and the actuator (not shown) such that the processor (or the remote software, or both) can communicate with and control the light arrays 128 (including the positioning and the actuation thereof) and the actuator for the conveyor 40 to operate in the fashion described herein. For example, the processor can use the known light output and distance from the bulbs to the object to calculate the necessary amount of time that the object much be exposed to the light. Such calculation can be performed either on a static basis or in real-time while the object is being transported through the device at a speed determined by the desired disinfection level. In certain examples, the conveyor is slowed or fully stopped to ensure disinfection. Once the required “dose” is achieved, the processor can initiate the actuation of the conveyor and thus the transport of the object out of the device.

Alternatively, the processor can be used for calculating the required time for the object to be disinfected to the desired level, regardless of whether the device 10 has a conveyor or not. That is, a device embodiment without a conveyor can have an indicator that can be coupled to the processor to indicate that the object can be manually removed from the device because the disinfection is complete. In one embodiment, the indicator is a light on the device 10 that either lights up or changes color (for red to green, for example) to indicate to a user that the user can remove the object. In a further alternative, as mentioned above, the processor can control the conveyor and actuate such conveyor to transport the object from the device 10 when the disinfection is complete.

Alternatively, each object can have an indicator disposed on the object that captures the date and time that the disinfection occurred or an RFID tag or other such device that can be tracked by the device such that the processor therein can track the objects that have been disinfected.

Thus, it is understood that any embodiment of the device 10 disclosed herein that provide for the functions described herein can be controlled manually, via automation, and/or using an onboard processor and/or remote software. It is further understood that any onboard processor can be any known processor for use in onsite equipment and any remote software can be any known software for use with the systems described herein to operate one or more devices as described herein.

In use, the device 10 or two or more devices 10 can be positioned at a desired location (such as, for example, near the checkout aisles of a retail store) such that the device(s) 10 are accessible for any objects to be disinfected. When preparing to disinfect an object, the object is positioned by a user in proximity with the entrance 22 such that the object is in contact with the conveyor 40. Once the object is placed at least partially on or in contact with the conveyor 40 or is disposed such that one or more sensors (not shown) detect the presence of the object, the processor (not shown) can communicate with the actuator (not shown) to cause the conveyor 40 to be actuated. Alternatively, the conveyor 40 can be actuated manually. The object is then transported through the device 10 such that the object is disinfected as described above.

In certain embodiments, certain sensors (not shown) are used to detect the presence of the object for purposes of actuating the conveyor 40 while other sensors (not shown) are provided to detect the distance between the object and the walls 14, 16, 18 for purposes of repositioning the light arrays 128 to the optimal distance from the object. Alternatively, no sensors are provided and these actions are accomplished manually. In a further alternative, the conveyor 40 is constantly operating whenever the device 10 is operating or powered up.

As mentioned above, the device 10 can be configured to receive any type of objects to be disinfected. For example, retail carts are discussed in detail herein. In a further alternative, the objects can be articles of clothing or other retail products that have been examined, “tried on,” or otherwise touched by customers but not sold, such that the products need to be disinfected before they can be placed on display for future customers. Thus, the specific size and dimensions of the device 10 can vary according to the size of the products or other objects to be disinfected. Such implementations can also include appropriately sized conveyors and/or other components as discussed herein.

In a further embodiment, the length of the device 10 can be increased to assist with preventing the light from the bulbs 30 from exiting the interior of the device 10 and putting nearby people at risk. That is, the length of the device 10 can be increased while the size of the arrays 28 is maintained such that the distance between the bulbs 30 and the entrance 22 and exit 24 is increased, thereby reducing the risk of the light “escaping” the interior of the device 10. In a further alternative, protective covers (not shown) can be disposed over the entrance 22 and exit 24 to block the light from exiting the device 10. Such covers can be plastic shields, curtains, or any other such covers (not shown).

In a further embodiment in which the products are articles of clothing on hangers, the device 10 can include a conveyor that is attached to the top wall 18 such that the hangers can be conveyed along such conveyor. For example, in one specific, non-limiting embodiment, the conveyor can be a rail that is represented by the conveyor rail 50B as shown in FIGS. 1A-1D. Such a conveyor rail 50B is similar to the type of known conveyors used in dry cleaning establishments and other similar businesses and can be used to transport hangers containing articles of clothing (not shown) through the device 10.

Although the various embodiments have been described with reference to preferred implementations, persons skilled in the art will recognize that changes may be made in form and detail without departing from the spirit and scope thereof.

1. A disinfection device comprising:
(a) a body comprising:
(i) a bottom wall;
(ii) two side walls coupled to the bottom wall;
(iii) a top wall coupled to the two side walls;
(iv) a first opening defined at a first end of the body; and
(v) a second opening defined at a second end of the body;
(b) at least one light array disposed on an interior surface of each of the two side walls and the top wall, wherein the at least one light array is positionable in relation to the interior surface between a retracted position and an extended position; and
(c) a conveyor disposed on the bottom wall, wherein the conveyor extends from the first opening to the second opening such that any object disposed on the conveyor is urged into the body through the first opening and out of the body through the second opening.

2. The disinfection device of claim 1, wherein the at least one light array comprises at least two UV light bulbs.

3. The disinfection device of claim 1, wherein the at least one light array comprises a first shield disposed on a first side of the at least one light array and a second shield disposed on a second side of the at least one light array.

4. (canceled)

5. The disinfection device of claim 1, further comprising at least one protective structure disposed on the interior surface of each of the two side walls and the top wall.

6. The disinfection device of claim 5, wherein the at least one protective structure comprises a bar disposed at a predetermined distance from the interior surface.

7. The disinfection device of claim 1, wherein the conveyor comprises:
(a) two conveyor rollers; and
(b) a conveyor belt disposed around the two conveyor rollers.

8. The disinfection device of claim 7, wherein the conveyor belt comprises ridges disposed on an outer surface of the conveyor belt.

9-19. (canceled)

20. The disinfection device of claim 1, further comprising a processor comprising disinfection software, the disinfection software configured to ensure full disinfection of an object disposed on the conveyor by controlling a speed of the conveyor and a position of the at least one light array.

21. The disinfection device of claim 20, wherein the disinfection software is further configured to ensure full disinfection of the object disposed on the conveyor based on the known distance.

22. A disinfection device comprising:
(a) a body comprising:
(i) a bottom wall;
(ii) two side walls coupled to the bottom wall;
(iii) a top wall coupled to the two side walls;
(iv) a first opening defined at a first end of the body; and
(v) a second opening defined at a second end of the body;
(b) at least one light array disposed on an interior surface of each of the two side walls and the top wall, wherein the at least one light array produces a light intensity;
(c) a conveyor disposed on the bottom wall, the conveyor comprising:
(i) two conveyor rollers; and
(ii) a conveyor belt moveably disposed around the two conveyor rollers,
wherein the conveyor belt is configured to move around the two conveyor rollers at a belt speed;
(d) a processor operably coupled to the conveyor; and
(e) disinfection software associated with the processor, the disinfection software configured to ensure full disinfection of an object disposed on the conveyor based on the light intensity and the belt speed by controlling the belt speed.

23. The disinfection device of claim 22, wherein the at least one light array is disposed on the interior surface such that the at least one light array is disposed at a known distance from the object disposed on the conveyor.

24. The disinfection device of claim 23, wherein the disinfection software is further configured to ensure full disinfection of the object disposed on the conveyor based on the known distance.

25. The disinfection device of claim 22, further comprising:
(a) a conveyor belt speed sensor associated with the conveyor and in communication with the processor; and
(b) a distance sensor associated with the body and in communication with the processor.

26. The disinfection device of claim 22, further comprising:
(a) at least one object presence sensor associated with the body and in communication with the processor; and
(b) an actuator operably coupled to the conveyor and in communication with the processor.

27. The disinfection device of claim 22, wherein the at least one light array comprises at least two UV light bulbs.

28. The disinfection device of claim 22, wherein the at least one light array is positionable in relation to the interior surface on which it is disposed.

29. The disinfection device of claim 28, wherein the disinfection software is configured to ensure full disinfection of the object based on the light intensity, the belt speed, and a position of the at least one light array by controlling the belt speed and the position of the at least one light array.

30. A disinfection device comprising:
(a) a body comprising:
(i) a bottom wall;
(ii) two side walls coupled to the bottom wall;
(iii) a top wall coupled to the two side walls;
(iv) a first opening defined at a first end of the body; and
(v) a second opening defined at a second end of the body;
(b) at least one light array moveably disposed on an interior surface of each of the two side walls and the top wall, wherein the at least one light array produces a light intensity;
(c) a conveyor disposed on the bottom wall, the conveyor comprising:
(i) two conveyor rollers; and
(ii) a conveyor belt moveably disposed around the two conveyor rollers,
wherein the conveyor belt is configured to move around the two conveyor rollers at a belt speed;
(d) a processor operably coupled to the conveyor;
(e) a conveyor belt speed sensor associated with the conveyor and in communication with the processor;
(f) a distance sensor associated with the body and in communication with the processor; and
(g) disinfection software associated with the processor, the disinfection software configured to ensure full disinfection of an object disposed on the conveyor based on the light intensity and the belt speed by controlling the belt speed.

31. The disinfection device of claim 30, wherein the disinfection software is configured to ensure full disinfection of the object based on the light intensity, the belt speed, and a position of the at least one light array by controlling the belt speed and the position of the at least one light array.

32. The disinfection device of claim 30, wherein the at least one light array is movable between a retracted position and an extended position.
